(19) United States
(12) Patent Application Publication
(10) Pub. No.: US 2009/0312316 A1
(43) Pub. Date: Dec. 17, 2009

(54) NOVEL COMPOUNDS

(76) Inventors: Barrie Wilkinson, Essex (GB); Steven Moss, Essex (GB); Ming Zhang, Essex (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/296,482

(22) PCT Filed: May 17, 2007
(86) PCT No.: PCT/EP2007/054801

§ 371 Date: Aug. 3, 2009

(30) Foreign Application Priority Data
May 19, 2006 (GB) 0609981.6

(51) Int. Cl. A61K 31/365; C07D 313/00; C07D 405/02; C07D 413/02; A61P 35/00; A61P 27/02; A61K 31/5377; A61K 31/4427

(52) U.S. Cl. 514/231.5; 549/271; 546/281.7; 544/147; 514/450; 514/336

(57) ABSTRACT

The present invention relates to derivatives of borrelidin that are useful in medicine, e.g. in the treatment of cancer or B-cell malignancies, or other diseases in which angiogenesis contributes to the pathology including ophthalmic disorders such as diabetic retinopathy as well as age related macular degeneration (AMD), corneal neovascularisation and retinopathy or prematurity. The present invention also provides methods for the production of these compounds and their use in medicine, in particular in the treatment and/or prophylaxis of cancer or B-cell malignancies and other diseases in which angiogenesis is implicated in the pathogenic process.

### BACKGROUND OF THE INVENTION

Borrelidin 1 (FIG. 1) is an 18-membered macrolide produced by several bacterial strains including, but not limited to, Streptomyces rochei ATCC23956, Streptomyces parvulus Tü113 and Streptomyces parvulus Tü4055. The gross structure of borrelidin was first elucidated in 1967 (Keller-Scheirlein, 1967), and was subsequently refined by detailed NMR analysis (Kuo et al., 1989). The absolute configuration of borrelidin was confirmed by X-ray crystallography (Anderson et al., 1989). Its co-identity as the antibiotic treponemycin has been verified (Maehr & Evans, 1987).

A number of groups have reported the synthesis of fragments of the borrelidin structure, and four independent total syntheses of borrelidin have been reported (Hanessian et al., 2003; Duffey et al., 2003; Nagamitsu et al., 2004; Vong et al., 2004). In addition the gene cluster responsible for the biosynthesis of borrelidin by Streptomyces parvulus Tü4055 has been identified, cloned and sequenced (WO 2004/058976; Olano et al., 2004a). Based on this the application of biosynthetic engineering techniques have allowed elucidation of the biosynthetic pathway leading to borrelidin and to the production of new analogues (WO 2004/058976; Olano et al., 2003; Olano et al., 2004b; Moss et al., 2006).

Borrelidin was first discovered due to its antibacterial activity (Berger et al., 1949), although this antibacterial activity extends only to a limited number of micrococci, and is not found against all common test bacteria. The mode of action in sensitive microorganisms involves selective inhibition of threonyl tRNA synthetase (Paetz & Nass, 1973; Ruan et al., 2005). Other activities against spirochetes of the genus Treponema (Singh et al., 1985; U.S. Pat. No. 4,759,928), against viruses (Dickinson et al., 1965), uses for the control of animal pests and weeds (DE 3607287) and use as an agricultural fungicide (DE 19835669; U.S. Pat. No. 6,193,964) have been reported. Additionally, recently, borrelidin has been reported to have antimalarial activity against drug resistant Plasmodium falciparum strains (Otoguro et al., 2003). Between all of these reports only two have reported any synthetically modified derivatives. The first of these describes the benzyl ester and its bis-O-(4-nitrobenzoyl) derivative (Berger et al., 1949). The second of these describes the borrelidin methyl ester, the methyl ester bis O-acetyl derivative, and the methyl ester Δ₁₄₋₁₅-dihydro-, Δ_{14-15,12-13}-tetrahydro-, and Δ_{14-15,12-13}-tetrahydro-C12-amino derivatives (Anderton & Rickards, 1965). No biological activity was reported for any of these compounds.

A recent disclosure of particular interest is the discovery that borrelidin displays anti-angiogenesis activity (Wakabayashi et al., 1997). Angiogenesis is the process of the formation of new blood vessels. Angiogenesis occurs only locally and transiently in adults, being involved in, for example, repair following local trauma and the female reproductive cycle. It has been established as a key component in several pathogenic processes including cancer, rheumatoid arthritis and diabetic retinopathy (Perrin et al., 2005). Its importance in enabling tumours to grow beyond a diameter of 1-2 cm was established by Folkman (Folkman, 1986), and is provoked by the tumour responding to hypoxia. In its downstream consequences angiogenesis is mostly a host-derived process, thus inhibition of angiogenesis offers significant potential in the treatment of cancers, avoiding the hurdles of other anticancer therapeutic modalities such as the diversity of cancer types and drug resistance (Matter, 2001). It is of additional interest that recent publications have described the functional involvement of tyrosinyl- and tryptophanyl-tRNA synthetases in the regulation of angiogenesis (Wakasugi et al., 2002; Otani et al., 2002).

In the rat aorta matrix culture model of angiogenesis, borrelidin exhibits a potent angiogenesis-inhibiting effect and also causes disruption of formed capillary tubes in a dose dependent manner by inducing apoptosis of the capillary-forming cells (Wakabayashi et al., 1997). Borrelidin inhibited capillary tube formation with an IC₅₀ value of 0.4 ng/mL (0.8 nM). In the same study, borrelidin was shown to possess anti-proliferative activity towards human umbilical vein endothelial cells (HUVEC) in a cell growth assay; the IC₅₀ value was measured at 6 ng/mL, which is 15-fold weaker than the anti-angiogenesis activity measured in the same medium. This anti-proliferative activity of borrelidin was shown to be general towards various cell lines, such as would be seen in a cancer cell growth inhibition assay. In addition to these data, the authors report that borrelidin inhibits protein synthesis in the cultured rat cells, a factor most probably linked to its ability to inhibit threonyl tRNA synthetase; however the IC₅₀ value for anti-angiogenesis activity (0.4 ng/mL) was 50-fold lower than that reported for inhibition of protein synthesis (20 ng/mL), indicating different activities of the compound. It is thus believed that the anti-proliferative activity may be linked to threonyl tRNA-synthetase inhibition and structural modification of the molecule to specifically alter the two different activities provides a means to improve therapeutic index. The inhibition of threonyl tRNA synthetase can be measured by a number of published methods, e.g. as published by Ruan et al (Ruan et al., 2005).

Borrelidin also displays potent inhibition of angiogenesis in vivo using the mouse dorsal air sac model (Funahashi et al., 1999), which examines VEGF-induced angiogenesis and is an excellent model for studying tumour-angiogenesis. Borrelidin was administered at a dose of 1.8 mg/kg by intraperitoneal injection and shown to significantly reduce the increment of vascular volume induced by WiDr cells, and to a higher degree than does TNP-470, which is a synthetic angiogenesis inhibitor in clinical trials. Detailed controls verified that these data are for angiogenesis inhibition and not inhibition of growth of the tumour cells. The authors also showed that borrelidin is effective for the inhibition of the formation of spontaneous lung metastases of B16-BL6 melanoma cells at the same dosage by inhibiting the angiogenic processes involved in their formation.

JP 9-227,549 and JP 8-173,167 confirm that borrelidin is effective against WiDr cell lines of human colon cancer, and also against PC-3 cell lines of human prostate cancer. JP 9-227,549 describes the production of borrelidin by Streptomyces rochei Mer-N7167 (Ferm P-14670) and its isolation from the resulting fermentation culture. In addition to borrelidin 1,12-desnitrile-12-carboxyl borrelidin 2 (presumably a biosynthetic intermediate or shunt metabolite), 10-desmethyl borrelidin 3 (presumably a biosynthetic analogue arising from the mis-incorporation of an alternative malonyl-CoA extender unit in module 4 of the borrelidin PKS), 11-epiborrelidin 4 and the C14,C15-cis borrelidin analogue 5 were described (see FIG. 1). Thus, JP 9-227,549 specifies borrelidin and borrelidin analogues wherein a nitrile or carboxyl group is attached the carbon skeleton at C12, and a hydrogen atom or lower alkyl group is attached to the carbon skeleton at C10.

Borrelidin was investigated in the 1960's for its pharmaceutical (anti-viral) potential but these efforts were discontinued due to toxicity, in particular its effect as a chemical sensitizer (Lumb et al, 1965, Dickinson et al., 1965).

WO 01/09113 discloses the preparation of borrelidin analogues that have undergone synthetic modification at the carboxylic acid moiety of the cyclopentane ring. The activity of these compounds was examined using endothelial cell proliferation and endothelial capillary formation assays in a similar manner to that described above. In general, modification of the carboxyl moiety improved the selectivity for inhibiting capillary formation: the major reason for this improvement in selectivity is through a decrease in the cell proliferation inhibition activity whereas the capillary formation inhibitory activity was altered to a much lower degree. Specifically, the borrelidin-morpholinoethyl ester showed a 60-fold selectivity index, the borrelidin-amide showed a 37-fold selectivity index, the borrelidin-(2-pyridyl)-ethyl ester showed a 7.5-fold selectivity index and the borrelidin-morpholinoethyl amide showed a 6-fold selectivity index, for the capillary formation inhibitory activity versus cell proliferation with respect to borrelidin. The capillary formation inhibitory activity of these and other borrelidin derivatives was verified using a micro-vessel formation assay. In addition, the authors showed that borrelidin weakly inhibited the propagation of metastatic nodules, after removal of the primary tumour, when using a Lewis lung adenocarcinoma model. However, the borrelidin-(3-picolylamide) derivative was reported to inhibit very considerably the increase of micrometastases in rats after intraperitoneal and also with per os administration at subtoxic doses. Similarly, using the colon 38 spleen liver model, the metastasis-forming ability of mouse colon adenocarcinoma cells transplanted into mouse spleen was considerably decreased after treatment with a subtoxic dose of this borrelidin derivative. These data confirm the earlier reported ability of borrelidin and its derivatives to inhibit the formation of metastases.

Borrelidin has also been identified as an inhibitor of cyclin-dependant kinase Cdc28/Cln2 of Saccharomyces cerivisiae with an IC₅₀ value of 12 μg/mL (24 μM) (Tsuchiya et al., 2001). It was shown that borrelidin arrests both haploid and diploid cells in late G₁ phase (at a time point indistinguishable from α-mating pheromone), and at concentrations that do not affect gross protein biosynthesis. These data were taken to indicate that borrelidin has potential as a lead compound to develop anti-tumour agents.

Two further reports have been published concerning the biological activity of borrelidin. The first of these indicates that the anti-angiogenic effects of borrelidin are mediated through distinct pathways (Kawamura et al., 2003). High concentrations of threonine were found to attenuate the ability of borrelidin to inhibit both capillary tube formation in the rat aorta culture model and HUVEC cells proliferation; however, it did not affect the ability of borrelidin to collapse formed capillary tubes or to induce apoptosis in HUVEC. Borrelidin was also found to activate caspase-3 and caspase-8, and inhibitors of both of these suppressed borrelidin induced apoptosis in HUVEC. The second of these papers used the method of global cellular mRNA profiling to provide insight into the effects of borrelidin on Saccharomyces cerevisiae (Eastwood and Schaus, 2003). This analysis showed the induction of amino acid biosynthetic enzymes in a time-dependent fashion upon treatment with borrelidin, and it was ascertained that the induction of this pathway involves the GCN4 transcription factor.

Certain analogues of borrelidin have been described in WO2004/058976 (Biotica Technology Ltd et al).

In summary, the angiogenesis-inhibitory effect of borrelidin is directed towards the twin biological effects of proliferation and capillary formation. In addition, borrelidin, and derivatives thereof, have been shown to inhibit the propagation of metastases. Borrelidin also has indications for use in cell cycle modulation. Thus, borrelidin and related compounds are particularly attractive targets for investigation as therapeutic agents for the treatment of tumour tissues, either as single agents or for use as an adjunct to other therapies. In addition, they may be used for treating other diseases in which angiogenesis is implicated in the pathogenic process, including, but not restricted to, the following list: rheumatoid arthritis, psoriasis, atherosclerosis, and various ophthalmic disorders such as diabetic retinopathy as well as age-related macular degeneration (AMD), corneal neovascularisation and retinopathy of prematurity.

The present invention provides derivatives of borrelidin which are useful as anticancer and anti-B-cell malignancy agents, and as agents for the treatment of other diseases in which angiogenesis is implicated in the pathogenic process.

Therefore, there remains a need to identify novel borrelidin derivatives with improved therapeutic index, which may have utility in the treatment of cancer and/or B-cell malignancies, or as agents for the treatment of other diseases in which angiogenesis is implicated in the pathogenic process. Preferably such borrelidin derivatives have one or more of the following properties: an improved ratio of anti-angiogenic activity to inhibition of general cell proliferation and/or tRNA synthetase activity, improved water solubility, improved cell permeability, an improved pharmacological profile and reduced side-effect profile for administration. The present invention discloses derivatives of borrelidin analogues with either open chain starter units or 4-membered ring starter units which generally have improved biological properties compared with borrelidin and related analogues; in particular they show improvements in respect of an improved ratio of anti-angiogenic activity to inhibition of general cell proliferation.

### SUMMARY OF THE INVENTION

The present invention provides derivatives of borrelidin, methods for the preparation of these compounds, intermediates thereto and methods for the use of these compounds in medicine.

In a more specific aspect the present invention provides derivatives of borrelidin according to the formula (I) below, or a pharmaceutically acceptable salt thereof:

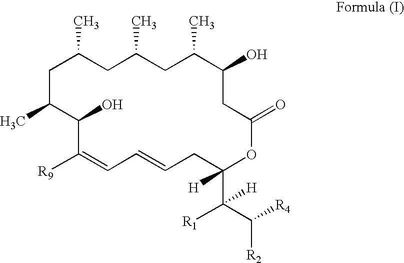

### Where:

R₁ and R₂ each independently represent H, SR₃ or a C1-C4 alkyl group which may be optionally substituted with one or more groups selected from OH, F, Cl or SR₃; where R₃ represents H, CH₃ or COCH₃; alternatively R₁ and R₂ together with the carbons to which they are joined represent a 4 membered cycloalkyl ring optionally substituted with one or more halo atoms or one or more C1 to C3 alkyl groups
R₄ represents

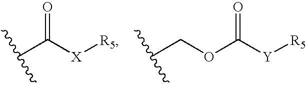

or —NHNHC(O)R₈ where R₈ represents biotin, H or a C1-C4 alkyl group optionally substituted with one or more groups selected from OH, F, Cl;
X represents —NH— or —O—;
Y represents —NH—, —O— or —CH₂—;
R₅ represents H or —(CH₂)ₙR₆, where:
n represents an integer between 1 and 3,
R₆ represents H, —OH, —OCH₃, —CO₂R₇, or a C1 to C4 alkyl group optionally substituted with one or more groups selected from OH, F, Cl, —CO₂R₇, —COR₇, where R₇ represents a C1-C4 alkyl group or R₆ represents:
   i) a 6 membered aromatic ring,
   ii) a 5 to 7 membered heteroaromatic ring containing between one and three N, S or O atoms,
   iii) a 5-7 member cycloalkyl group or
   iv) a 5-7 membered heteroalkyl ring containing between one and three N, S or O atoms,
each of i) to iv) above may be optionally substituted with one or more groups selected from CH₃, OCH₃, F, Cl or Br
R₉ represents CN, CO₂H, CH₃ or CONH₂;
provided, however, that when X represents —O— then R₅ does not represent H.

The above structure shows a representative tautomer and the invention embraces all tautomers of the compounds of formula (I) for example keto compounds where enol compounds are illustrated and vice versa.

In a further aspect, the present invention provides borrelidin derivatives such as compounds of formula (I) or a pharmaceutically acceptable salt thereof, for use as a pharmaceutical.

### Definitions

The articles “a” and “an” are used herein to refer to one or to more than one (i.e. at least one) of the grammatical objects of the article. By way of example “an analogue” means one analogue or more than one analogue.

As used herein the term “analogue(s)” refers to chemical compounds that are structurally similar to another but which differ slightly in composition (as in the replacement of one atom by another or in the presence or absence of a particular functional group).

In particular, the term “borrelidin analogue” refers to a borrelidin compound produced by the methods of WO 2004/058976 and as shown by formula (II). These compounds are also referred to as “parent compounds” and these terms are used interchangeably in the present application. In the present application the term “borrelidin analogues” includes reference to borrelidin itself.

As used herein, the term “borrelidin derivative” refers to a borrelidin derivative referred to above as representing the invention in its broadest aspect, i.e. a compound according to formula (I) above, or a pharmaceutically acceptable salt thereof. These compounds are also referred to as “compounds of the invention” or “derivatives of borrelidin” and these terms are used interchangeably in the present application.

As used herein, the term “cancer” refers to a malignant new growth that arises from epithelium, found in skin or, more commonly, the lining of body organs, for example, breast, prostate, lung, kidney, pancreas, stomach or bowel. A cancer tends to infiltrate into adjacent tissue and spread (metastasise) to distant organs, for example to bone, liver, lung or the brain. As used herein the term cancer includes both metastatic tumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, gliobastoma, primary liver cancer and ovarian cancer.

As used herein the term “B-cell malignancies” includes a group of disorders that include chronic lymphocytic leukaemia (CLL), multiple myeloma, and non-Hodgkin's lymphoma (NHL). They are neoplastic diseases of the blood and blood forming organs. They cause bone marrow and immune system dysfunction, which renders the host highly susceptible to infection and bleeding.

The pharmaceutically acceptable salts of compounds of the invention such as the compounds of formula (I) include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. References hereinafter to a compound according to the invention include both compounds of formula (I) and their pharmaceutically acceptable salts.

Alkyl, alkenyl and alkynyl groups may be straight chain or branched.

Examples of C1-C4 alkyl groups include methyl, ethyl, n-propyl, i-propyl and n-butyl.

5 to 7-membered cycloalkyl groups refer to a cycloalkyl ring including 5-7 carbon atoms that may optionally be branched. Examples include cyclopentyl and cyclohexyl.

5 to 7 membered heteroalkyl rings containing one or more heteroatoms selected from N, O and S include rings containing one or two heteroatoms, especially one heteroatom. Examples include furan, pyran, oxetane, oxirane, piperidine, pyrrolidine, azetidine, aziridine, thiirane, thiethane, thiophene, thiopyran and morpholine.

6-membered aromatic rings include phenyl.

5 to 7-membered heteroaromatic rings containing 1 to 3 heteroatoms selected from O, N and S include 6-membered rings such as pyridyl and pyrimidinyl and 5-membered rings such as furanyl, pyrrolyl, imidazolyl, thiophenyl, oxazolyl, oxadiazolyl, thiazolyl and thiadiazolyl.

### DESCRIPTION OF THE INVENTION

The present invention provides derivatives of borrelidin, as set out above, methods for the preparation of these compounds, intermediates thereto and methods for the use of these compounds in medicine.

Suitably R₉ represents CN.

Suitably R₁ and R₂ together with the carbons to which they are joined represent a 4 membered cycloalkyl ring.

Suitably R₁ and R₂ together with the carbons to which they are joined represent a 4 membered cycloalkyl ring, only substituted by R₄.

Suitably X represents —O—. Alternatively, suitably X represents —NH—.

Suitably Y represents —NH— or—O—. In one embodiment Y represents —NH—. In another embodiment Y represents —O—.

Suitably R₄ represents

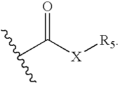

Suitably n represents 1. Alternatively, suitably n represents 2.

Suitably R₆ represents a 6 membered heteroaromatic ring containing between one and three N, S or O atoms.

More suitably R₆ represents a 6 membered heteroaromatic ring containing one N atom.

Most suitably R₆ represents

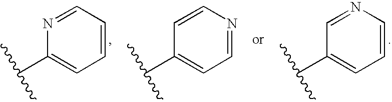

Alternatively, suitably R₆ represents a 6-membered heteroalkyl ring containing between one and three N, S or O atoms

More suitably R₆ represents

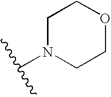

Suitably, R₁ and R₂ together with the carbons to which they are joined represent a 4 membered cycloalkyl ring, substituted only with R₄ which represents

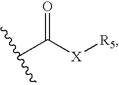

where X represents —O— and R₅ represents —(CH₂)ₙR₆, where n represents 2 and R₆ represents

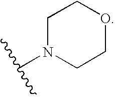

Alternatively, suitably R₁ and R₂ together with the carbons to which they are joined represent a 4 membered cycloalkyl ring substituted only with R₄ which represents

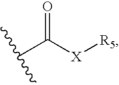

where X represents —O— and R₅ represents —(CH₂)ₙR₆, where n represents 2 and R₆ represents

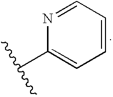

Alternatively, suitably, R₁ and R₂ together with the carbons to which they are joined represent a 4 membered cycloalkyl ring substituted only with R₄ which represents

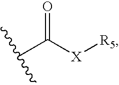

where X represents —NH— and R₅ represents —(CH₂)ₙR₆, where n represents 2 and R₆ represents

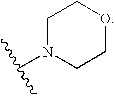

Alternatively, suitably R₁ and R₂ together with the carbons to which they are joined represent a 4 membered cycloalkyl ring substituted only with R₄ which represents

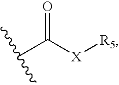

where X represents —NH— and R₅ represents —(CH₂)ₙR₆, where n represents 1 and R₆ represents

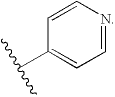

Alternatively, suitably R₁ and R₂ together with the carbons to which they are joined represent a 4 membered cycloalkyl ring substituted only with R₄ which represents

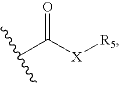

where X represents —NH— and R₅ represents —(CH₂)ₙR₆, where n represents 1 and R₆ represents

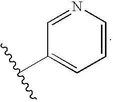

Alternatively, suitably R₁ and R₂ together with the carbons to which they are joined represent a 4 membered cycloalkyl ring substituted only with R₄ which represents

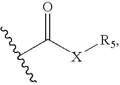

where X represents —NH— and R₅ represents —(CH₂)ₙR₆, where n represents 1 and R₆ represents

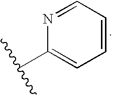

Alternatively, suitably R₁ and R₂ together with the carbons to which they are joined represent a 4 membered cycloalkyl ring substituted only with R₄ which represents

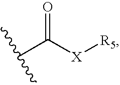

where X represents —NH— and R₅ represents —(CH₂)ₙR₆, where n represents 2 and R₆ represents

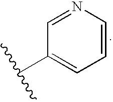

Alternatively, suitably R₁ and R₂ together with the carbons to which they are joined represent a 4 membered cycloalkyl ring substituted only with R₄ which represents

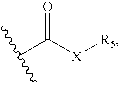

where X represents —NH— and R₅ represents H.

In general, the compounds of the invention are prepared by semi-synthetic derivatisation of a borrelidin analogue. Borrelidin analogues may be prepared using methods as described in WO 2004/058976, which document is incorporated herein by reference in its entirety.

In general, the process for preparing a compound of formula (I) or a pharmaceutically acceptable salt thereof comprises:
(a) reacting a compound of formula (II):

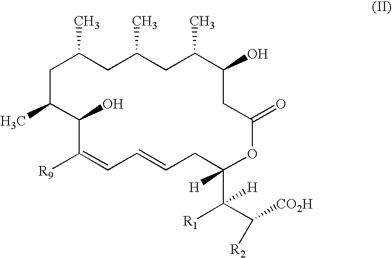

or a protected derivative thereof, using any one of the methods described in (i) to (viii) below, or
(b) converting a compound of formula (I) or a salt thereof to another compound of formula (I) or another pharmaceutically acceptable salt thereof; or
(c) deprotecting a protected compound of formula (I).

Compounds of formula (II) may be suitably be produced by any methods known to a person of skill in the art. In particular, according to the methods described in WO 2004/058976.

The present invention provides methods for preparing the compounds of formula (I), in particular using esterifying, amidating and reducing methods known to a person of skill in the art. In addition to the specific methods and references provided herein a person of skill in the art may also consult standard textbook references for synthetic methods, including, but not limited to Vogel's Textbook of Practical Organic Chemistry (Furniss et al., 1989) and March's Advanced Organic Chemistry (Smith and March, 2001).

The compounds of general formula (I) can be prepared by adapting the general methods described herein, for example by using, but not limited to, the following processes:
(i) reaction of an acid chloride formed from a compound of formula (II) with a suitable alcohol or amine;
(ii) direct esterification or amidation of a compound of formula (II) in the presence of carbodiimide, such as N-(3-dimethylaminopropyl)-N′-ethylcarbodiimide (EDCI), and a base;
(iii) transesterification of an ester, formed from a compound of formula (II), with a suitable alcohol;
(iv) reaction of a methyl ester of a compound of formula (II) with a suitable amine;
(v) formation of an active ester from a compound of formula (II), e.g. with 1-hydroxybenztriazole, then reaction with a suitable alcohol or amine;
(vi) formation of a mixed anhydride from a compound of formula (II), e.g. with chloroformic acid ester, then reaction with a suitable amine;
(vii) reduction of a mixed anhydride from a compound of formula (II) with a metal hydride to an alcohol;
(viii) alkylation and acylation of a primary alcohol prepared from a compound of formula (II).

In particular ester derivatives of a compound of formula (II) can be most preferably prepared by reacting an activated derivative formed from a compound of formula (II) with 1-hydroxybenztriazole in the presence of carbidiimide (such as N-(3-dimethylaminopropyl)-N′-ethylcarbodiimide (EDCI)), with a suitable alcohol.

Such a reaction is typically carried out in inert solvents, such as but not limited to tetrahydrofuran (THF). In such a reaction N-(3-dimethylaminopropyl)-N′-ethylcarbodiimide (EDCI) is used as the carbodiimide and dimethylaminopyridine (DMAP) is used as a base. It is suitable to use a 10-fold molar excess of the alcohol component. The reaction is carried out at a temperature between 0° C. and 100° C., preferably at ambient (e.g. 25° C.), with stirring for between 1 and 10 h, but typically for 3 h.

Amide derivatives of a compound of formula (II) can be readily prepared, for example with the mixed anhydride derivative formed chloroformic acid ester. The reaction can be carried out in inert organic and water-free solvents such as, but not limited to, THF or dichloromethane (DCM). Triethylamine, pyridine and 4-(dimethylamino)pyridine can be used as base. Typically the amine to be coupled can be present in up to 10-fold molar excess. The reaction is carried out by stirring at a temperature between −20° C. and 50° C. The reaction is typically left to stir for between 1 and 10 h. In a preferred embodiment the of the process the mixed anhydride is formed with isobutyl chloroformate in anhydrous THF at −20° C., in the presence of triethylamine, and coupling is performed over 3 h after adding between 5- and 10-fold excess of the amine.

Primary alcohol derivatives of a compound of formula (II) can be prepared from a mixed anhydride and reduction with complex metal hydride in THF at −20° C. The alkylation and acylation of such compounds can be carried out by any general method, such as those described in Vogel's Textbook of Practical Organic Chemistry (Furniss et al., 1989) and March's Advanced Organic Chemistry (Smith and March, 2001).

In addition to the specific methods and references provided herein a person of skill in the art may also consult standard textbook references for synthetic methods, including, but not limited to Vogel's Textbook of Practical Organic Chemistry (Furniss et al, 1989) and March's Advanced Organic Chemistry (Smith and March, 2001).

In processes (a) and (c), examples of protecting groups and the means for their removal can be found in T W Greene “Protective Groups in Organic Synthesis” (J Wiley and Sons, 1991). Suitable hydroxyl protecting groups include alkyl (e.g. methyl), acetal (e.g. acetonide) and acyl (e.g. acetyl or benzoyl) which may be removed by hydrolysis, and arylalkyl (e.g. benzyl) which may be removed by catalytic hydrogenolysis, or silyl ether, which may be removed by acidic hydrolysis or fluoride ion assisted cleavage. Suitable amine protecting groups include sulphonyl (e.g. tosyl), acyl (e.g. benzyloxycarbonyl or t-butoxycarbonyl) and arylalkyl (e.g. benzyl) which may be removed by hydrolysis or hydrogenolysis as appropriate.

Other compounds of the invention may be prepared by methods known per se or by methods analogous to those described above.

The compounds of the invention are useful directly, and as templates for further semi-synthesis or bioconversion, to produce compounds useful as anticancer agents. Methods for the semi-synthetic derivatisation of borrelidin have been described in WO 01/09113.

The above structures of intermediates (e.g. compounds of formula (II)) may be subject to tautomerisation and where a representative tautomer is illustrated it will be understood that and all tautomers for example keto compounds where enol compounds are illustrated and vice versa are intended to be referred to.

The invention additionally provides for the use of a compound of the invention in the treatment of cancer or B-cell malignancies. It also provides a compound of the invention for use in the treatment of cancer or B-cell malignancies. It also provides a method of treatment of cancer or B-cell malignancies which comprises administering to a patient an effective amount of a compound of the invention. It also provides the use of a compound of the invention in the preparation of a medicament for the treatment of cancer or B-cell malignancies.

Borrelidin is also known to have utilities in the treatment of other conditions, including, but not limited to bacterial infections, viral infections and malaria and in other diseases in which angiogenesis is implicated in the pathogenic process, including, but not restricted to, the following: rheumatoid arthritis, psoriasis, atherosclerosis, diabetic retinopathy and various ophthalmic disorders. The uses and methods involving the compounds of the invention also extend to these other indications.

In a preferred embodiment, the present invention provides compounds with utility in the treatment of cancer or B-cell malignancies. One skilled in the art would be able by routine experimentation to determine the ability of these compounds to inhibit tumour cell growth(see for example the methods described in Roth et al., 1999 and Dengler et al., 1995 and the methods described in the Examples herein).

The present invention also provides a pharmaceutical composition comprising an ansamycin derivative, or a pharmaceutically acceptable salt thereof, together with a pharmaceutically acceptable carrier.

The aforementioned compounds of the invention or a formulation thereof may be administered by any conventional method for example but without limitation they may be administered parenterally (including intravenous administration), orally, topically (including buccal, sublingual or transdermal), via a medical device (e.g. a stent), by inhalation, or via injection (subcutaneous or intramuscular). The treatment may consist of a single dose or a plurality of doses over a period of time.

Whilst it is possible for a compound of the invention to be administered alone, it is preferable to present it as a pharmaceutical formulation, together with one or more acceptable carriers. Thus there is provided a pharmaceutical composition comprising a compound of the invention together with one or more pharmaceutically acceptable diluents or carriers. The diluents(s) or carrier(s) must be “acceptable” in the sense of being compatible with the compound of the invention and not deleterious to the recipients thereof. Examples of suitable carriers are described in more detail below.

The compounds of the invention may be administered alone or in combination with other therapeutic agents. Co-administration of two (or more) agents may allow for significantly lower doses of each to be used, thereby reducing the side effects seen. There is also provided a pharmaceutical composition comprising a compound of the invention and a further therapeutic agent together with one or more pharmaceutically acceptable diluents or carriers.

In a further aspect, the present invention provides for the use of a compound of the invention in combination therapy with a second agent for the treatment of cancer or B-cell malignancies.

In one embodiment, a compound of the invention is co-administered with another therapeutic agent for the treatment of cancer or B-cell malignancies preferred agents include, but are not limited to, methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Iressa®, Tarceva™, Erbitux™), HSP90 inhibitors (e.g. 17-(allylamino)-17-demethoxygeldanamycin (17-AAG) or 17-(dimethylaminoethylamino)-17-demethoxygeldanamycin (17-DMAG)), mTOR inhibitors (e.g. rapamycin, CCI-779, RAD001) or VEGF inhibitors (e.g. Avastin™), proteasome inhibitors (e.g. Velcade™) or Glivec®. Additionally, a compound of the invention may be administered in combination with other therapies including, but not limited to, radiotherapy or surgery.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

It should be understood that in addition to the ingredients particularly mentioned above the formulations of this invention may include other agents conventional in the art having regard to the type of formulation in question, for example those suitable for oral administration may include flavouring agents.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The compounds of the invention may also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The dosage to be administered of a compound of the invention will vary according to the particular compound, the disease involved, the subject, and the nature and severity of the disease and the physical condition of the subject, and the selected route of administration. The appropriate dosage can be readily determined by a person skilled in the art.

The compositions may contain from 0.1% by weight, preferably from 5-60%, more preferably from 10-30% by weight, of a compound of invention, depending on the method of administration.

It will be recognized by one of skill in the art that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

### BRIEF DESCRIPTION OF THE FIGURES

FIG. 1: illustrates the structure of borrelidin and some related metabolites isolated from borrelidin producing organisms

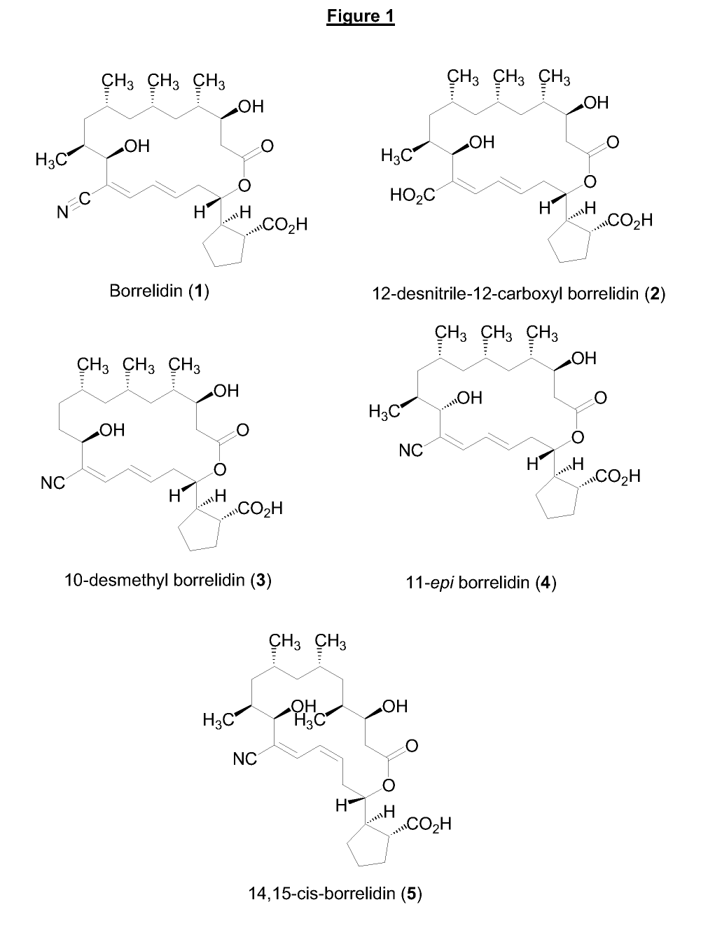

FIG. 2: shows the ¹H NMR spectra for 17-des(cyclopentane-2-carboxylic acid)-17-(cyclobutane-2-carboxylic acid)borrelidin (4-(2-hydroxyethyl))morpholine ester (7)

FIG. 3: shows the ¹H NMR spectra for 17-des(cyclopentane-2-carboxylic acid)-17-(cyclobutane-2-carboxylic acid)borrelidin (2-(2-hydroxyethyl)pyridine ester (8)

FIG. 4: shows the ¹H NMR spectra for 17-des(cyclopentane-2-carboxylic acid)-17-(cyclobutane-2-carboxylic acid)borrelidin (4-(2-aminoethyl))morpholine amide (9)

FIG. 5: shows the ¹H NMR spectra for 17-des(cyclopentane-2-carboxylic acid)-17-(cyclobutane-2-carboxylic acid)borrelidin (4-aminomethyl)pyridine amide (10)

FIG. 6: shows the ¹H NMR spectra for 17-des(cyclopentane-2-carboxylic acid)-17-(cyclobutane-2-carboxylic acid)borrelidin (3-aminomethyl)pyridine amide (11)

FIG. 7: shows the ¹H NMR spectra for 17-des(cyclopentane-2-carboxylic acid)-17-(cyclobutane-2-carboxylic acid)borrelidin (2-aminomethyl)pyridine amide (12)

FIG. 8: shows the ¹H NMR spectra for 17-des(cyclopentane-2-carboxylic acid)-17-(cyclobutane-2-carboxylic acid)borrelidin (3-(2-aminoethyl))pyridine amide (13)

FIG. 9: shows the ¹H NMR spectra for 17-des(cyclopentane-2-carboxylic acid)-17-(cyclobutane-2-carboxylic acid)borrelidin amide (14)

FIG. 10: Structures of the compounds (6-14) produced in the examples.

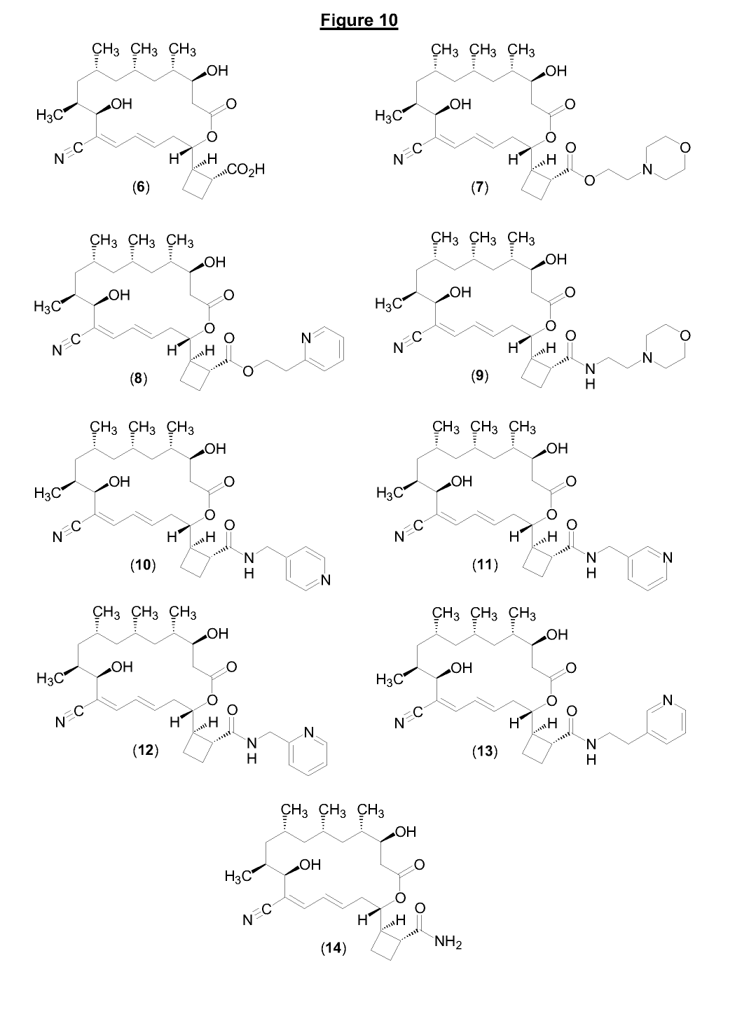

### EXAMPLES

### General Methods

### Fermentation of Cultures

The production of borrelidin analogues was performed as described previously in WO 2004/058976 and a typical example is provided below (Example 1).

LCMS Analysis Procedure for Fermentation Broth Analysis and in vivo Transformation Studies

Culture broth (1 mL) and ethyl acetate (1 mL) were mixed for 15 min followed by centrifugation for 10 min. 0.5 mL of the organic layer was collected, evaporated to dryness and then re-dissolved in 0.25 mL of methanol. LCMS was performed on an integrated Agilent HP1100 HPLC system in combination with a Bruker Daltonics Esquire 3000+ electrospray mass spectrometer operating in positive ion mode. Chromatography was achieved over a Phenomenex Hyperclone column (C₁₈ BDS, 3 u, 150×4.6 mm) eluting over 25 min at a flow rate of 1 mL/min with a linear gradient from water+0.1% formic acid to acetonitrile+0.1% formic acid.

### Synthesis

All reactions were conducted under anhydrous conditions unless stated otherwise, in oven dried glassware that was cooled under vacuum, and using dried solvents. Reactions were monitored by LC-UV-MS on Micromass Platform, single quadrapole instrument.

Chromatography was achieved over reversed-phase silica using an Atlantis dC18 column (50×2.1 mm, 5 micron) eluting at 1 ml/min with the following gradient: T=0, 100% A; T=2.5, 100% B; mobile phase A, water+0.1% formic acid; mobile phase B, acetonitrile+0.1% formic acid. UV detection was performed at 215 nm.

### Assessment of Water Solubility

Water solubility may be tested as follows: A 10 mM stock solution of the Borrelidin analogue is prepared in 100% DMSO at room temperature. Triplicate 0.01 mL aliquots are made up to 0.5 mL with either 0.1 M PBS, pH 7.3 solution or 100% DMSO in amber vials. The resulting 0.2 mM solutions are shaken in the dark, at room temperature on an IKA® vibrax VXR shaker for 6 h, followed by transfer of the resulting solutions or suspensions into 2 mL Eppendorf tubes and centrifugation for 30 min at 13200 rpm. Aliquots of the supernatant fluid are then analysed by the LCMS method as described above.

### Assessment of Cell Permeability

Cell permeability may be tested as follows: The test compound is dissolved to 10 mM in DMSO and then diluted further in buffer to produce a final 10 μM dosing concentration. The fluorescence marker lucifer yellow is also included to monitor membrane integrity. Test compound is then applied to the apical surface of Caco-2 cell monolayers and compound permeation into the basolateral compartment is measured. This is performed in the reverse direction (basolateral to apical) to investigate active transport. LC-MS/MS is used to quantify levels of both the test and standard control compounds (such as Propanolol and Acebutolol).

### NMR Structure Determination of Synthetic Compounds

¹H NMR spectra were recorded at 400 MHz on a Bruker 400 machine. Compounds were dissolved in CDCl₃ and referenced to the residual proton resonating at δ=7.26 ppm.

### In Vitro Bioassay for Anticancer Activity

In vitro evaluation of compounds for anticancer activity in a panel of 12 human tumour cell lines in a monolayer proliferation assay was carried out at the Oncotest Testing Facility, Institute for Experimental Oncology, Oncotest GmbH, Freiburg. The characteristics of the 12 selected cell lines are summarised in Table I.

TABLE I
Test cell lines

| # | Cell line | Characteristics |
|---|---|---|
| 1 | MCF-7 | Breast, NCI standard |
| 2 | MDA-MB-231 | Breast - PTEN positive, resistant to 17-AAG |
| 3 | MDA-MB-468 | Breast - PTEN negative, resistant to 17-AAG |
| 4 | NCI-H460 | Lung, NCI standard |
| 5 | SF-268 | CNS, NCI standard |
| 6 | OVCAR-3 | Ovarian - p85 mutated. AKT amplified. |
| 7 | A498 | Renal, high MDR expression, |
| 8 | GXF 251L | Gastric |
| 9 | MEXF 394NL | Melanoma |
| 10 | UXF 1138L | Uterus |
| 11 | LNCAP | Prostate - PTEN negative |
| 12 | DU145 | Prostate - PTEN positive |

The Oncotest cell lines were established from human tumor xenografts as described by Roth et al., (1999). The origin of the donor xenografts was described by Fiebig et al., (1999). Other cell lines were either obtained from the NCI (H460, SF-268, OVCAR-3, DU145, MDA-MB-231, MDA-MB-468) or purchased from DSMZ, Braunschweig, Germany (LNCAP).

All cell lines, unless otherwise specified, were grown at 37° C. in a humidified atmosphere (95% air, 5% CO₂) in a ‘ready-mix’ medium containing RPMI 1640 medium, 10% fetal calf serum, and 0.1 mg/mL gentamicin (PAA, Cölbe, Germany).

### Monolayer Assay—Brief Description of Protocol:

A modified propidium iodide assay was used to assess the effects of the test compound(s) on the growth of human tumour cell lines (Dengler et al., (1995)).

Briefly, cells were harvested from exponential phase cultures by trypsinization, counted and plated in 96 well flat-bottomed microtitre plates at a cell density dependent on the cell line (5-10.000 viable cells/well). After 24 h recovery to allow the cells to resume exponential growth, 0.010 mL of culture medium (6 control wells per plate) or culture medium containing macbecin were added to the wells. Each concentration was plated in triplicate. Compounds were applied in two concentrations (0.001 mM and 0.01 mM). Following 4 days of continuous exposure, cell culture medium with or without test compound was replaced by 0.2 mL of an aqueous propidium iodide (PI) solution (7 mg/L). To measure the proportion of living cells, cells were permeabilized by freezing the plates. After thawing the plates, fluorescence was measured using the Cytofluor 4000 microplate reader (excitation 530 nm, emission 620 nm), giving a direct relationship to the total number of viable cells.

The mean growth inhibition across the 12 cell-line panel was expressed as treated/control×100 (% T/C).

The activity of borrelidin analogues shown on this cancer cell growth inhibition assay is believed to occur largely via inhibition of threonyl tRNA synthetase.

### Example 1

### Fermentation and isolation of 17-des(cyclopentane-2-carboxylic acid)-17-(cyclobutane-2-carboxylic acid)borrelidin (6)

The example given is specific for the production of 6 but the method is generally to all borrelidin analogues, a person of skill in the art will appreciate how to adapt the specific method described below for the production of alternative borrelidin analogues. 3×7 L Applikon fermentors were run as described and the fermentation broths combined for extraction.

A seed flask containing NYG medium (30 mL in a 250 mL Erlenmyer flask) was inoculated with S. parvulus Tü4055/borG::aac3(IV)) (WO 2004/058976) from a mycelial stock culture (0.5 mL; generated by 1:1 dilution of a 2 day seed culture with cryo-preservative (20% w/v glycerol and 10% w/v lactose in deionised water)). NYG medium contains, in deionised water: beef extract (0.3% w/v), Bacto peptone (0.5% w/v), glucose (1% w/v) and yeast extract (0.5% w/v).

Secondary seeds were also prepared as above (but with 250 mL NYG in 2 L Erlenmyer flasks). PYDG production medium (4 L) with 0.01% v/v Pluronic L0101 to prevent foaming was inoculated with the secondary seed inocula (10% v/v). PYDG medium contains, in deionised water: peptonised milk nutrient (1.5% w/v), yeast autolysate (0.15% w/v), dextrin (4.5% w/v) and glucose (0.5% w/v) adjusted to pH 7.0. This was allowed to ferment in a 7 L Applikon fermenter for 6 days at 30 ° C. After 24 h cyclobutane 1,2-trans-dicarboxylic acid in methanol was added to give a final concentration of 4 mM. Airflow was set at 0.75 vvm, with tilted baffles and the impeller speed controlled electronically between 250 and 600 rpm in order to maintain dissolved oxygen tension at or above 30% of air saturation. The fermentation broth of three such fermenters was combined (12 L) and assayed; a total of 339 mg of 6 was produced (at an average titre 28 mg/L).

The fermentation broth was adjusted to pH˜5.5 (conc. HCl) and then clarified by centrifugation. The supernatant was extracted with ethylacetate (2×1 volume equivalent), and the cell pellet extracted with methanol (2×1 volume equivalent). The combined organic extracts were evaporated to yield a brown aqueous slurry. This was diluted with water (to 1 L) and extracted with ethylacetate (3×1 L). The combined organic extracts were evaporated to yield brown oil (˜4.5 g). The oil was dissolved into acetone, flash silica gel 60 was added (150 g) and the solvent removed by evaporation. This was applied to a flash silica column (5×25 cm) pre-prepared in heptane. The column was eluted with a stepped gradient from 100% heptane to 100% ethyl acetate. Fractions containing 6 were identified by TLC, combined, and the solvent removed by evaporation to give a yellow oil (˜1 g). This oil was then dissolved in the minimum volume of solvent mixture (heptane:CHCl₃:ethanol, 10:10:1) and applied to a column of Sephadex LH-20 (2.5×60 cm) pre-prepared in the same solvent mixture. The column was eluted under gravity with approx. 3 L of the same solvent mixture, collecting 12 mL fractions. Fractions containing 6 were combined and the solvent removed by evaporation. The resulting pale yellow solid (550 mg) was crystallised by dissolving into a minimum amount of CHCl₃ and then adding hexane until it became cloudy. The resulting solution was stored at −20° C. overnight and the resulting solid collected by filtration and then washed with cold hexane. The mother liquor was treated similarly again. The two batches were combined to yield 6 (190 mg).

NMR spectra for 6 were recorded on a Bruker Advance 500 spectrometer at 298 K operating at 500 MHz and 125 MHz for ¹H and ¹³C respectively. Standard Bruker pulse programs were used to acquire the ¹H-¹H COSY, APT, HMQC and HMBC spectra; coupling constants are given in hertz. NMR experiments were run in CDCl₃ and were referenced to the residual proton resonating at δ_{H} 7.26 and carbon at δ_{C} 77.0 for CDCl₃.

TABLE II

| | |
|---|---|
| | (6) |
| 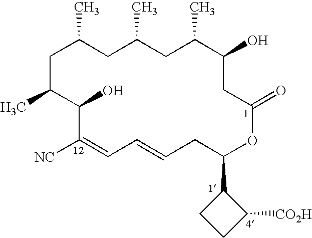 | |

| Position | | δ_{C} | δ_{H} (mult., Hz) | H-H COSY | H-C HMBC |
|---|---|---|---|---|---|
| 1 | O—C═O | 172.3 | — | — | — |
| 2 | CH₂ | 34.3 | 2.35 dd (16.7, 2.0) | 3 | 1, 3, 4 |
| | | | 2.47 dd (16.7, 10.3) | | |
| 3 | CH—OH | 70.0 | 3.89 dt (10.4, 2.2) | 2, 4 | 1, 4, 5, 18 |
| 4 | CH | 35.7 | 1.61 m | 3, 5a, 18 | |
| 5 | CH₂ | 43.1 | 1.24 ddd (10.4, 10.4, 2.2) | 4, 6 | 3, 4, 7, 18, 19 |
| | | | 0.90 ddd (19.0, 8.3, 2.7) | | |
| 6 | CH | 27.4 | 1.54 m | 5, 7, 19 | |
| 7 | CH₂ | 47.6 | 1.12 ddd | 6 | 5, 8, 9, 19, 20 |
| | | | 0.96 m | 8 | |
| 8 | CH | 26.2 | 1.60 m | 7, 9, 20 | |
| 9 | CH₂ | 37.4 | 1.04 m | 8, 9b, 10 | 8, 10, 11, 20, 21 |
| | | | 0.71 td (14.3, 3.1) | 8, 9a | |
| 10 | CH | 35.2 | 1.86 m | 9a, 11, 21 | |
| 11 | CH—OH | 73.1 | 4.11 d (9.7) | 10 | 9, 10, 12, 13, 21 |
| 12 | C═ | 118.2 | — | — | — |
| 13 | CH═ | 144.0 | 6.80 d (11.3) | 14 | 11, 12, 15, 22 |
| 14 | CH═ | 126.9 | 6.39 dd (14.9, 11.5) | 13, 15 | 13, 22 |
| 15 | CH═ | 138.5 | 6.10 ddd (8.9, 3.4, 3.0) | 14, 16 | 13 17 |
| 16 | CH₂ | 34.3 | 2.46 m | 15, 17 | 15, 17, 1′ |
| 17 | CH—O | 75.5 | 5.11 m | 16, 1′ | 1′, 2′, 1, 15 |
| 18 | 4-CH₃ | 17.0 | 0.84 d (6.7) | 4 | 3, 4, 5 |
| 19 | 6-CH₃ | 18.2 | 0.79 d (6.3) | 6 | 5 |
| 20 | 8-CH₃ | 20.2 | 0.82 d (5.2) | 8 | 7, 8, 9 |
| 21 | 10-CH₃ | 14.9 | 1.04 d (6.4) | 10 | 9, 11 |
| 22 | —CN | 115.9 | — | — | — |
| 1′ | CH | 40.2 | 2.89 quint (9.0) | 17, 2′, 4′ | 2′, 4′, 5′, 17 |
| 2′ | CH | 41.9 | 3.08 qrad (9.0) | 1′, 3′ | 1′, 3′, 5′, 17 |
| 3′ | CH₂ | 21.5 | 2.15 m | 2′, 4′ | 1′ |
| 4′ | CH₂ | 21.1 | 2.02 m | 1′, 3′ | 1′, 3′, 17 |
| | | | 1.76 quint (9.6) | | |
| 5′ | —CO₂H | 177.3 | — | — | — |

ESI-MS m/z: 498.4 [M + Na]⁺, 476.3 [M + H]⁺, 458.4 [M − 18]⁺; 474.5 [M − H]⁻, 456.4 [M − 18]⁻
UV: λₘₐₓ (DAD) = 258 nm

### Example 2

### Synthesis of 17-des(cyclopentane-2-carboxylic acid)-17-(cyclobutane-2-carboxylic acid)borrelidin (4-(2-hydroxyethyl))morpholine ester (7)

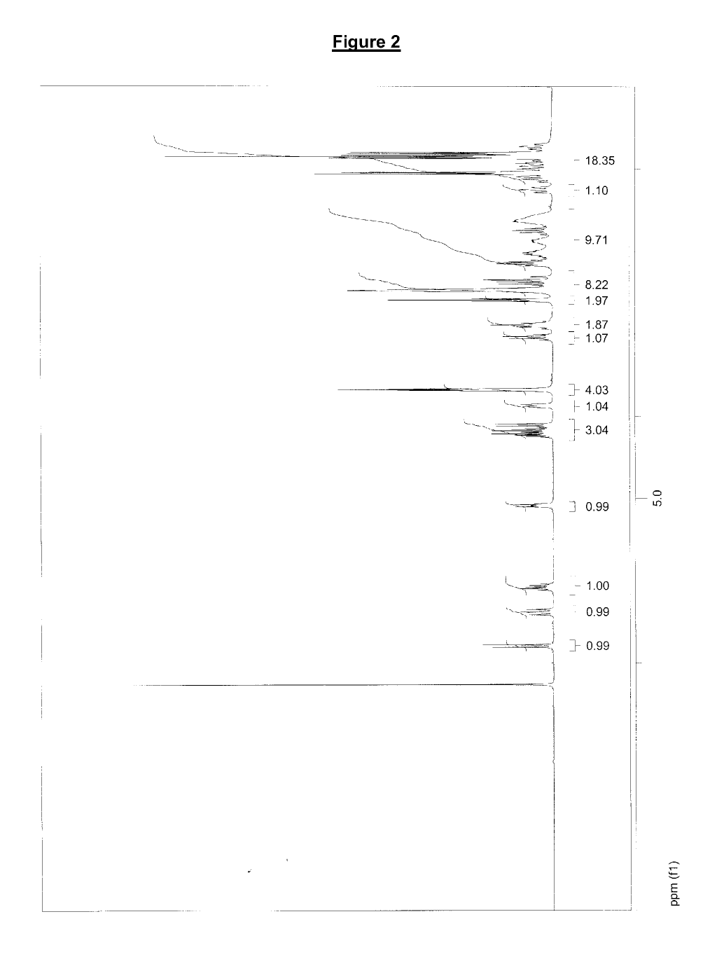

A stirred solution of borrelidin analogue 6 (125 mg, 0.263 mmol) in anhydrous tetrahydrofuran (THF) (7 mL) was treated under nitrogen with 1-hydroxytriazole (35 mg, 0.263 mmol), N-(3-dimethylaminopropyl)-N′-ethylcarbodiimide (EDCI) (64 mg, 0.33 mmol) and DMAP (73 mg, 0.60 mmol). After 30 min 4-(2-hydroxyethyl)morpholine (344 mg, 0.32 mL, 2.63 mmol) was added. After a further 3 h the mixture was evaporated to dryness. The residue was taken up in dichloromethane (DCM), washed twice with water and dried over magnesium sulphate. After filtration and evaporation the residue was purified by ‘Combiflash’ eluting with a gradient of 0-90% ethyl acetate in DCM. The product was evaporated to dryness and azeotroped twice with chloroform to yield a colourless glass. Yield=61 mg, 39%. Purity (by LCMS) >95%. ¹H NMR was consistent with the correct compound (FIG. 2).

### Example 3

### Synthesis of 17-des(cyclopentane-2-carboxylic acid)-17-(cyclobutane-2-carboxylic acid)borrelidin (2-(2-hydroxyethyl)pyridine ester (8)

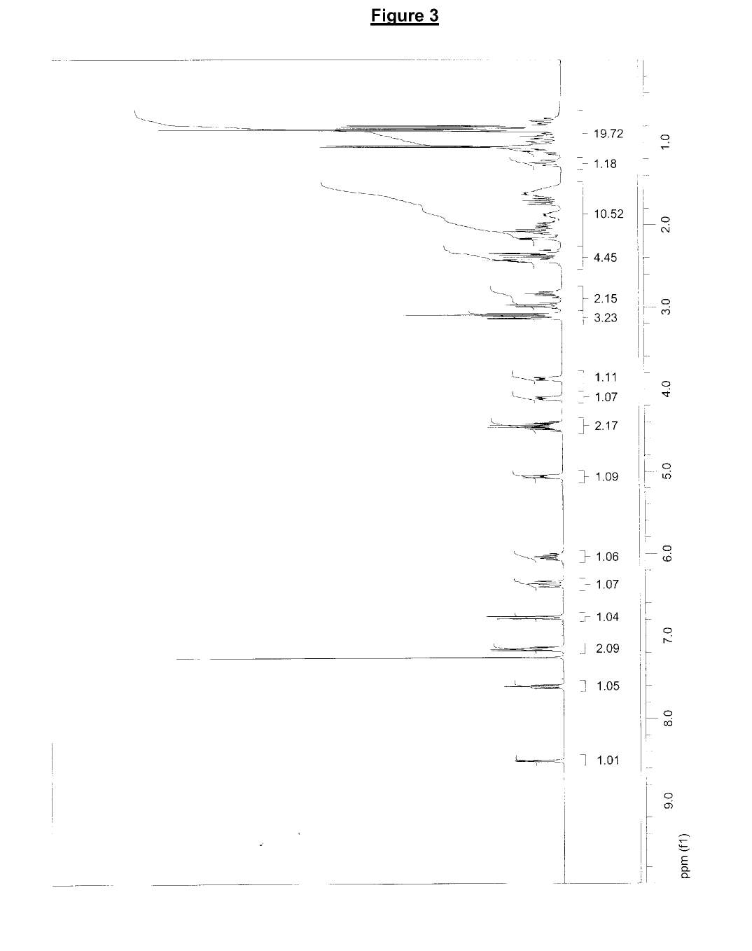

This compound was prepared as described in example 2 using the same amounts of material but with 2-(2-hydroxyethyl)pyridine (323 mg, 0.30 mL, 2.63 mmol) in place of the 4-(2-hydroxyethyl)morpholine. Yield=89 mg, 58%. Purity (by LCMS) >95%. ¹H NMR was consistent with the correct compound (FIG. 3).

### Example 4

### Synthesis of 17-des(cyclopentane-2-carboxylic acid)-17-(cyclobutane-2-carboxylic acid)borrelidin (4-(2-aminoethyl))morpholine amide (9)

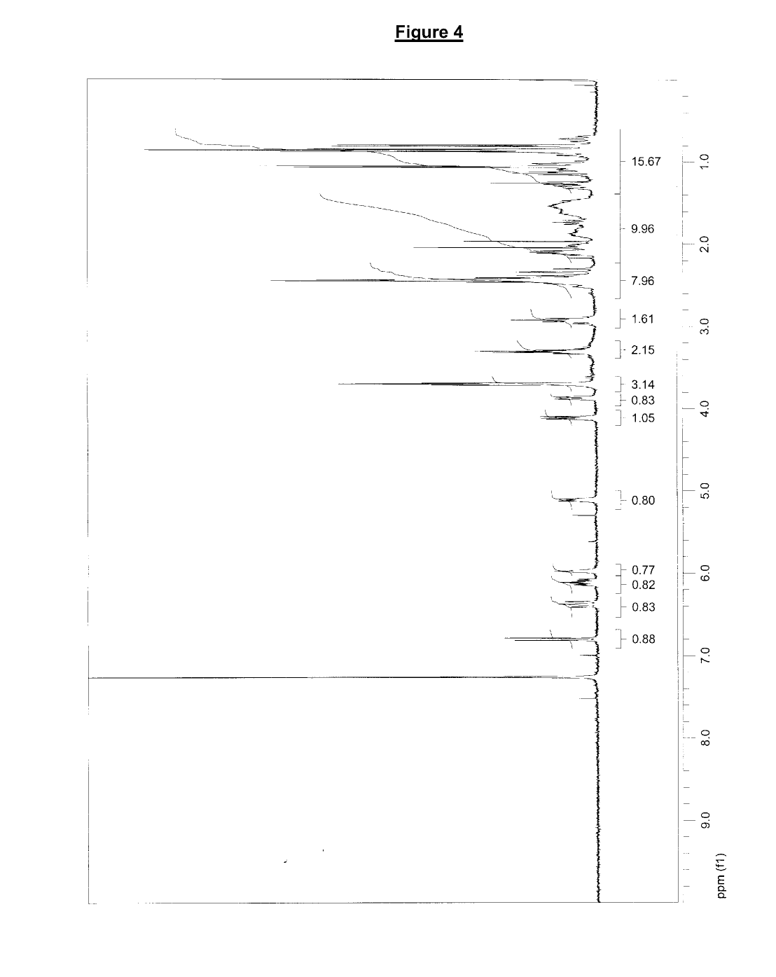

A stirred solution of 6 (100 mg, 0.204 mmol) in dry THF (7.5 mL) was cooled to −20° C. and treated, under nitrogen, with dry triethylamine (0.03 mL, 0.225 mmol) followed by isobutyl chloroformate (0.03 mL, 0.225 mmol). After stirring at −20° C. for 30 min the mixture was filtered to remove triethylamine hydrochloride. The filtrate was again cooled to −20° C. and treated, under nitrogen, with 4-(2-aminoethyl)morpholine (0.17 mL, 1.3 mmol). The temperature was then allowed to rise to ambient over the next 3 h, after which time the mixture was evaporated to dryness. The residue was taken up in DCM, washed three times with water and dried over magnesium sulphate. After filtering the solvent was removed evaporation at room temperature. The product was purified by ‘Combiflash’ eluting with a gradient of 0-90% ethyl acetate in DCM, followed by 5% triethylamine in ethyl acetete. After evaporation the product was obtained as a faintly yellow glass. Yield=84 mg, 54%. Purity (by LCMS) >99%. ¹H NMR was consistent with the correct compound (FIG. 4).

### Example 5

### Synthesis of 17-des(cyclopentane-2-carboxylic acid)-17-(cyclobutane-2-carboxylic acid)borrelidin (4-aminomethyl)pyridine amide (10)

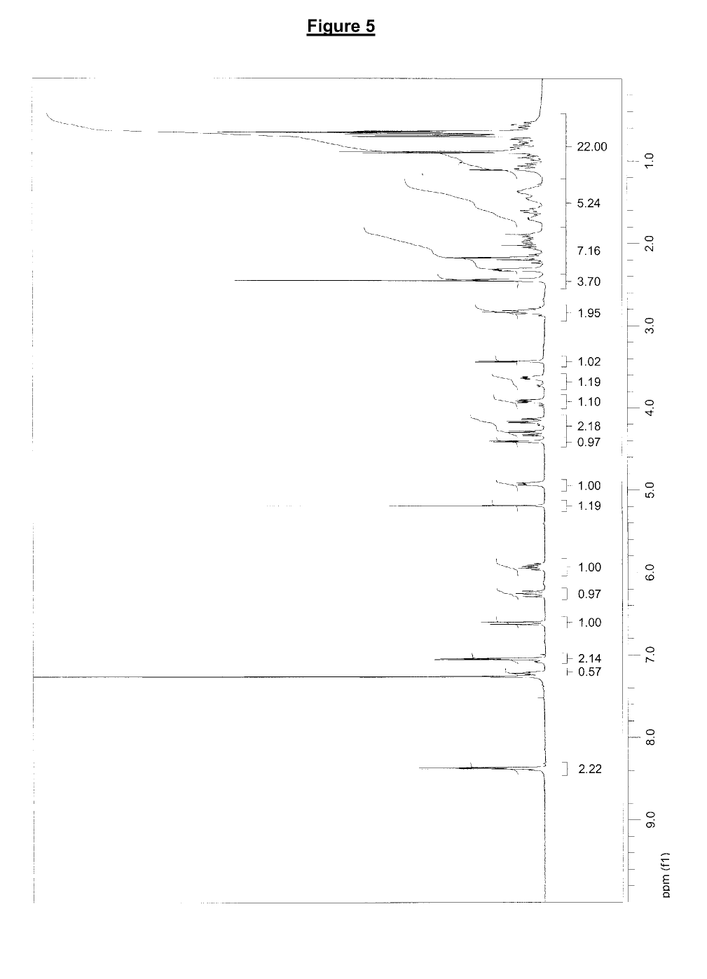

This compound was prepared as described in example 4 but using the following amounts of material: borrelidin analogue 6 (125 mg, 0.263 mmol), dry THF (10 mL), dry triethylamine (29 mg, 0.04 mL, 0.289 mmol), isobutyl chloroformate (39 mg, 0.04 mL, 0.289 mmol) and 4-(aminomethyl)pyridine (181 mg, 0.17 mL, 1.68 mmol). Purification was performed using ‘Combiflash’ using 0-90% ethyl acetate in DCM, followed by neat ethyl acetate. The product was isolated as a white solid. Yield=32 mg, 21.5%. Purity (by LCMS) >97%. ¹H NMR was consistent with the correct compound but required the addition of some d₆-DMSO to aid solubility of the product (FIG. 5).

### Example 6

### Synthesis of 17-des(cyclopentane-2-carboxylic acid)-17-(cyclobutane-2-carboxylic acid)borrelidin (3-aminomethyl)pyridine amide (11)

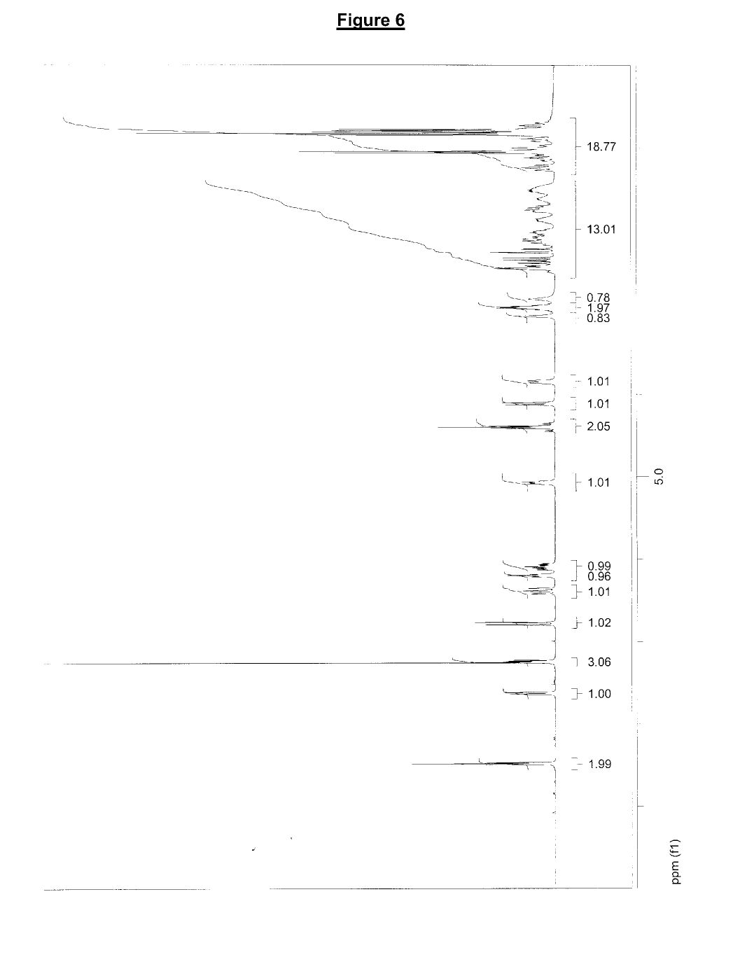

This compound was prepared as described in example 4 using the same amounts of material but with 3-(aminomethyl)pyridine (181 mg, 0.17 mL, 1.68 mmol) in place of 4-(2-aminoethyl)morpholine. Purification was performed using ‘Combiflash’ using 0-90% ethyl acetate followed by 90% ethyl acetate in DCM (the product eluted after a by-product). The product was azeotroped twice with chloroform to give a colourless glass. Yield=121 mg, 81%. Purity (by LCMS) 100%. ¹H NMR was consistent with the correct compound (FIG. 6).

### Example 7

### Synthesis of 17-des(cyclopentane-2-carboxylic acid)-17-(cyclobutane-2-carboxylic acid)borrelidin (2-aminomethyl)pyridine amide (12)

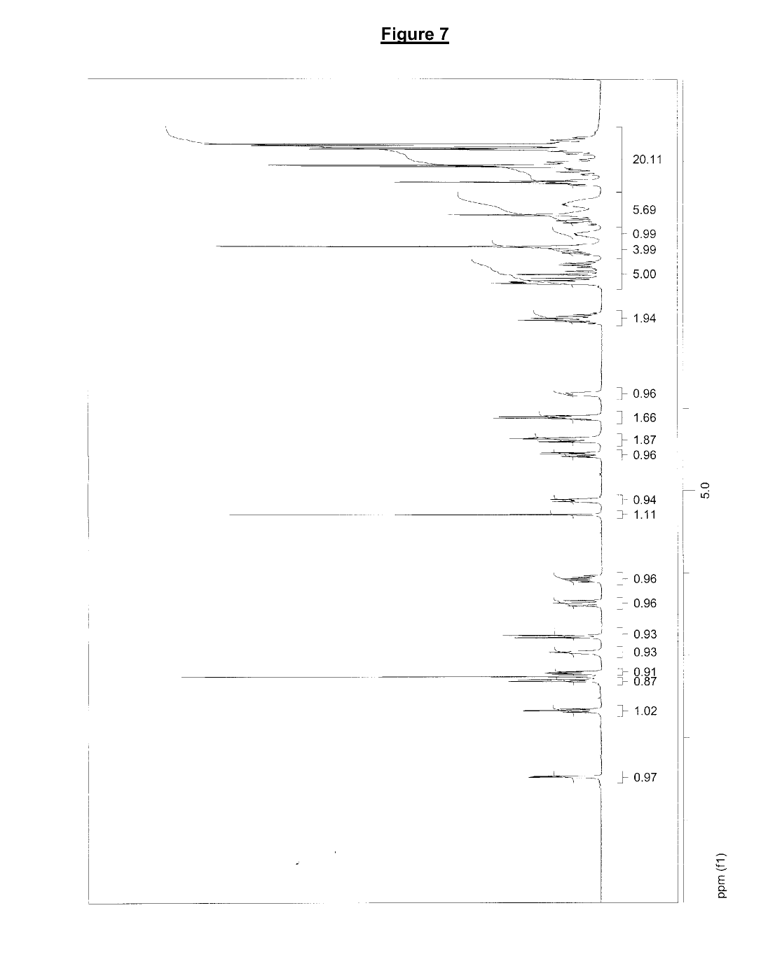

This compound was prepared as described in example 4 using the same amounts of material but with 2-(aminomethyl)pyridine (181 mg, 0.17 mL, 1.68 mmol) in place of 4-(2-aminoethyl)morpholine. Purification as example 4 to give the product as a colourless glass. Yield=137 mg, 92%. Purity (by LCMS) 100%. ¹H NMR was consistent with the correct compound (FIG. 7).

### Example 8

### Synthesis of 17-des(cyclopentane-2-carboxylic acid)-17-(cyclobutane-2-carboxylic acid)borrelidin (3-(2-aminoethyl))pyridine amide (13)

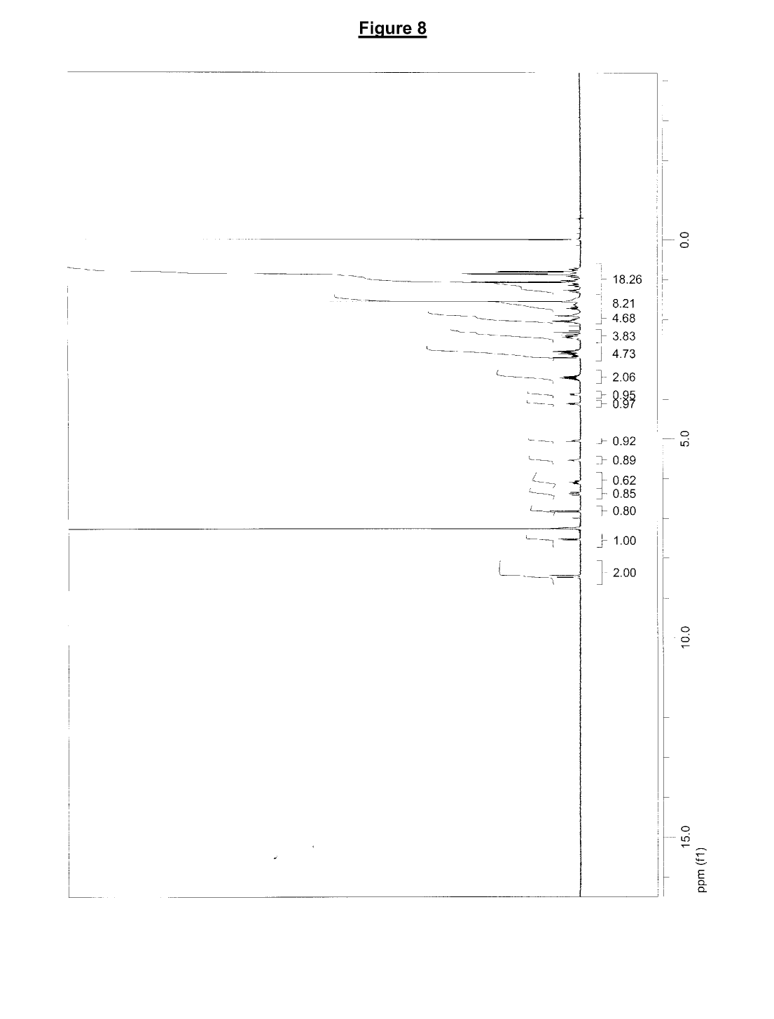

This compound was prepared as described in example 4 using the same amounts of material but with 3-(2-aminoethyl)pyridine (205 mg, 0.27 mL, 1.68 mmol) in place of 4-(2-aminoethyl)morpholine. Purification as example 4 to give the product as a colourless glass. Yield=98 mg, 64%. Purity (by LCMS) 100%. ¹H NMR was consistent with the correct compound (FIG. 8).

### Example 9

### Synthesis of 17-des(cyclopentane-2-carboxylic acid)-17-(cyclobutane-2-carboxylic acid)borrelidin amide (14)

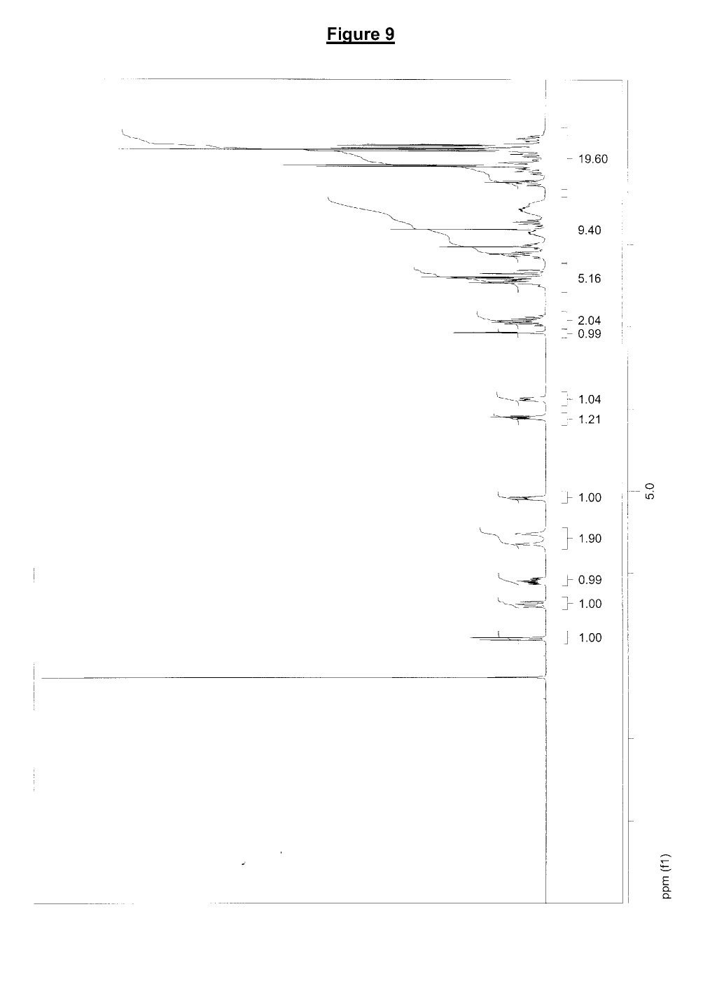

This compound was prepared as described in example 4 using the same amounts of material but with aqueous ammonia (0.1 mL) in place of 4-(2-aminoethyl)morpholine. Three drops of glacial acetic acid were added immediately before workup and then purification as example 4 to give the product as a colourless glass. Yield=90 mg, 72%. Purity (by LCMS) >99%. ¹H NMR was consistent with the correct compound (FIG. 9).

### Example 10

### Cleavage studies of ester derivatives 7 & 8 and Amide Derivatives 9 & 13

Compounds 7, 8, 9 & 13 were incubated in rat blood and examined for their cleavage to give the parent acid 6.

Compounds 7, 8, 9 & 13 were added to rat blood (containing EDTA) at a final concentration of 0.01 mg/mL and incubated at 37° C. Samples were taken at time 2, 30, 60, 120 and 180 minutes and extracted using SPE cartridges. Briefly, sample (0.1 mL) was transferred to a 2 mL propylene tube and internal standard was added. The sample was diluted to a volume of 1 mL by addition of 0.1 M aqueous KH₂PO₄. The sample was vortexed for a period of 5 min and centrifuged at 3000 rpm for 5 min. An Oasis MAX cartridge (1 cc/30 mg) was conditioned with 1 mL MeOH and 1 mL water successively. The supernatant was transferred to the conditioned cartridge at a flow rate of 1-2 mL/min. The cartridge was washed with 2% NH₄OH in water and 30% MeOH (containing 2% formic acid) at 1-2 mL/min. Finally, the analyte was eluted with 80% MeOH (containing 2% formic acid) at 1-2 mL/min. The extracts were transferred to autosampler vials and immediately analysed by LC/MS. Samples were compared to a standard calibration curve for 6.

The time 2 minute samples for each compound were serially diluted after extraction and analysed by LC-MS to ensure that their response was linear over a concentration range of 3-100%. LCMS was performed on a Bruker Daltonics Esquire 3000+ mass spectrometer equipped with an electrospray source coupled to the HPLC. The HPLC conditions were: 20% B for 1 min followed by a linear gradient to 100%B over a period of 7 min and an isocratic period of 2 min at 100% B. Injection volume: 0.05 mL. HPLC was performed on a Waters Symmetry C8 3.5 micron column, 50 mm×2.1 mm, running a mobile phase of: mobile phase A: 0.1% formic acid in water; mobile phase B: 0.1% Formic acid in acetonitrile; flow rate: 1 mL/minute.

Compound 6 shows an intense [M−H]⁻ ion at m/z 474. Fragmentation of this molecular species results in a spectrum with intense ions at m/z 271 and 412. The daughter ion at m/z 412 can be ideally selected for quantification.

Using these methods the amide derivatives 9 & 13 did not cleave to give any of the parent compound 6. The two ester derivatives 7 & 8 rapidly cleaved to give the parent compound 6. The half-life for the cleavage of 7 was estimated at 12 min, and for 8 the cleavage rate was too rapid to calculate using this protocol.

Based on these data the ester derivatives 7 and 8 are considered to act as potential pro-drugs of 6. The amide derivatives do not function as pro-drugs of 6.

### Example 11

### Biological Data—In Vitro Evaluation of Anticancer Activity of the Borrelidin Derivatives

In vitro evaluation of the test compounds for anticancer activity in a panel of human tumour cell lines in a monolayer proliferation assay was carried out as described in the general methods using a modified propidium iodide assay. This screen may be used as a model for the toxic effect of compounds in inhibiting general cell growth.

The results are displayed in Table III below; each result represents the mean of triplicate experiments, except for compounds 1 and 6 for which the results represent the mean of duplicate experiments

TABLE III
in vitro cell line data

Mean % T/C

| Compound # | at 1 μM | at 10 μM |
|---|---|---|
| 1 | 7 | 6 |
| 6 | 33 | 8 |
| 7 | 36 | 8 |
| 8 | 40 | 6 |
| 9 | 101 | 43 |
| 10 | 104 | 57 |
| 11 | 97 | 20 |
| 12 | 87 | 15 |
| 13 | 97 | 37 |
| 14 | 92 | 26 |

Compounds 9, 10, 11, 12, 13 and 14 resulted in little or no inhibition of cell growth at 1 μM and therefore these compounds can be said to have a relatively good safety profile. Compounds 7 and 8 were improved relative to compound 6 itself improved relative to compound 1.

### Example 12

### Biological Data—Evaluation of Anti-Angiogenic Activity of Borrelidin Derivatives Using a Chick Chorioallantoic Membrane (CAM) Assay

Fresh fertile eggs were be incubated for 3 days in a standard egg incubator at 37° C. for 3 days. On Day 3, eggs were cracked under sterile conditions and embryos were placed into 20×100 mm plastic plates and cultivated at 37° C. in an embryo incubator with a water reservoir on the bottom shelf. Air was continuously bubbled into the water reservoir using a small pump so that the humidity in the incubator was kept constant. On Day 6, a sterile silicon “o” ring was placed on each CAM and a set amount of test compound dissolved in 10 μl of 0.5% methylcellulose was delivered into each “o” ring in a sterile hood. Embryos were returned to the incubator after addition of test material. Control embryos received 10 μL of vehicle alone. On Day 8, embryos were removed from the incubator and kept at room temperature while blood vessel density was determined under each “o” ring using an image capturing system at a magnification of 160×.

The blood vessel density was semi-quantitatively measured using an angiogenesis scoring system, in that exponential numbers 1 to 32 were used to indicate number of blood vessels present at the treatment sites on the CAM. Number 32 represented the highest density and 0 represented no angiogenesis. The percent of inhibition at each dosing site was calculated using the score recorded for that site divided by the mean score obtained from the appropriate control samples for each individual experiment. The percent of inhibition for each dose of a given compound was calculated by pooling all results obtained for that dose.

The results are displayed in Table IV below; each mean blood vessel density score represents the mean of ten experiments. The percentage inhibition of cancer cell lines at 1 uM was calculated by subtracting the mean %T/C at 1 uM from table III from 100. A higher value in the ratio of inhibition of CAM vasculature to in vitro cancer cell inhibition would suggest a relative increase in antiangiogenic activity compared to general cell growth inhibition, or tRNA synthetase activity.

TABLE IV
CAM assay data and ratio of cancer cell inhibition to angiogenesis inhibition

| | | | | Ratio of inhibition of |
|---|---|---|---|---|
| | | Mean percentage | | CAM vasculature at |
| | | inhibition of CAM | Mean percentage | 25 ng/CAM to in vitro |
| | Mean blood vessel | vasculature at | Inhibition of cancer | cancer cell inhibition |
| Compound # | density score | 25 ng/CAM | cell lines at 1 uM | at 1 uM |
| 1 | 6.4 | 62 | 93 | 0.6 |
| 6 | 8.2 | 51 | 67 | 0.76 |
| 7 | 7.8 | 54 | 64 | 0.84 |
| 9 | 6.8 | 60 | 0 | >60 |

From this data, it would seem that the anti-angiogenic therapeutic index (ratio of anti-angiogenesis to general cell inhibition) of 9 is especially good, and 7 and 9 are improved, as compared to 1 and 6.

### REFERENCES

Anderson, B. F., Herlt, A. J., Rickards, R. W., and Robertson, G. B. (1989) Crystal and molecular structures of two isomorphous solvates of the macrolide antibiotic borrelidin: absolute configuration determination by incorporation of a chiral solvent in the in the crystal lattice. Aust. J. Chem. 42:717-730.
Anderton, K. and R. W. Rickards (1965). “Some structural features of borrelidin, an anti-viral antibiotic.” Nature 206(981): 269.
Berger, J., Jampolsky, L. M., and Goldberg, M. W. (1949) Borrelidin, a new antibiotic with anti-Borrelia activity and penicillin enhancement properties. Arc. Biochem. 22:476-478.
Dengler W. A., Schulte J., Berger D. P., Mertelsmann R. and Fiebig H H. (1995) Development of a propidium iodide fluorescence assay for proliferation and cytotoxicity assay. Anti-Cancer Drugs, 6:522-532.
Dickinson, L., Griffiths, A. J., Mason, C. G., and Mills, R. F. (1965) Anti-viral activity of two antibiotics isolated from a species of Streptomyces. Nature 206:265-268.
Duffey, M. O., A. LeTiran, et al. (2003). “Enantioselective total synthesis of borrelidin”, J Am Chem Soc 125(6): 1458-1459.
Eastwood, E. L. and S. E. Schaus (2003). “Borrelidin induces the transcription of amino acid biosynthetic enzymes via a GCN4-dependent pathway.” Bioorg Med Chem Lett 13(13): 2235-7.
Folkman, J. (1986) How is blood vessel growth regulated in normal and neoplastic tissue? G.H.A. Cloves Memorial Lecture. Cancer Res. 51:467-473.
Funahashi, Y., Wakabayashi, T., Semba, T., Sonoda, J., Kitoh, K., and Yoshimatsu, K. (1999) Establishment of a quantitative mouse dorsal air sac model and its application to evaluate a new angiogenesis inhibitor. Oncol. Res. 11:319-329.
Furniss B. S., Hannaford A. J., Smith P. W. G. and Tatchell A. R. (1989) Vogel's textbook of practical organic chemistry, 5^{th} Ed, Pearson, Prentice Hall, Harlow, UK
Hanessian, S., Y. Yang, et al. (2003). “Application of conformation design in acyclic stereoselection: total synthesis of borrelidin as the crystalline benzene solvate.” J Am Chem Soc 125(45): 13784-92.
Kawamura, T., D. Liu, et al. (2003). “Anti-angiogenesis effects of borrelidin are mediated through distinct pathways: threonyl-tRNA synthetase and caspases are independently involved in suppression of proliferation and induction of apoptosis in endothelial cells.” J Antibiot (Tokyo) 56(8): 709-15.
Keller-Schierlein, W. (1967). “[Metabolic products of microorganisms. Concerning the constitution of borrelidin].” Helv Chim Acta 50(3): 731-53.
Kuo, M. S., D. A. Yurek, et al. (1989). “Assignment of ¹H and ¹³C NMR signals and the alkene geometry at C-7 in borrelidin.” Journal of Antibiotics 42(6): 1006-1007.
Lumb, M., Macey, P. E., Spyvee, J., Whitmarsh, J. M. and Wright, R. D. (1965). “Isolation of vivomycin and borrelidin, two antibiotics with anti-viral activity, from a species of Streptomyces.” Nature 206: 263-265.
Maehr, H. and R. H. Evans (1987). “Identity of borrelidin with treponemycin.” Journal of Antibiotics 40(10): 1455-1456.
Matter, A,. (2001) Tumor angiogenesis as a therapeutic target. Drug Dis. Today 6:1005-1024.
Moss, S. J., I. Carletti, C. Olano, R. M. Sheridan, M. Ward, V. Math, M. Nur-E-Alam, A. F. Braña, M. Q. Zhang, P. F. Leadlay, C. Méndez, J. A. Salas and B. Wilkinson. (2006) “Biosynthesis of the angiogenesis inhibitor borrelidin: directed biosynthesis of novel analogues”. Chem. Commun., in press.
Nagamitsu, T., Takano, D., Fukuda, T., Otoguro, K., Kuwajima, I., Harigaya, Y. and Ōmura, S. (2004) Total synthesis of (−)-borrelidin. Org. Lett., 6, 1865-1867.
Olano C, Wilkinson B, Moss S J, Brana A F, Mendez C, Leadlay P F, Salas J A. (2003), “Evidence from engineered gene fusions for the repeated use of a module in a modular polyketide synthase.” Chem. Commun, 2780-2782.
Olano C, Wilkinson B, Sanchez C, Moss S J, Sheridan R, Math V, Weston A J, Brana A F, Martin C J, Oliynyk M, Mendez C, Leadlay P F, Salas J A. (2004a) “Biosynthesis of the angiogenesis inhibitor borrelidin by Streptomyces parvulus Tu4055: cluster analysis and assignment of functions.” Chem. Biol., 11, 87-97.
Olano C, Moss S J, Brana A F, Sheridan R M, Math V, Weston A J, Mendez C, Leadlay P F, Wilkinson B, Salas J A. (2004b) “Biosynthesis of the angiogenesis inhibitor borrelidin by Streptomyces parvulus Tu4055: insights into nitrile formation.” Mol. Microbiol., 52, 1745-1756.
Otani, A., Slike, B. M., Dorrell, H. I., Hood, J., Kinder, K., Cheresh, D. A., Schimmel, P., and Friedlander, M. (2002) A fragment of human TrpRS as a potent antagonist of ocular angiogenesis. Proc. Nat. Acad. Sci. USA 99:178-183.
Otoguru, K., Ui, H., Ishiyama, A., Kobayashi, M., Togashi, H., Takahashi, Y., Masuma, R., Tanaka, H., Tomado, H., Yamada, H., and Ōmura, S. (2003) In vitro and in vivo antimalarial activities of a non-glycosidic 18-membered macrolide antibiotic, borrelidin, against drug-resistant strains of Plasmodia. J. Antibiot. 56:727-729.
Paetz, W., and Nass, G. (1973) Biochemical and immunological characterization of threonyl-tRNA synthetase of two borrelidin-resistant mutants of Escherichia coli K12. Eur. J. Biochem. 35:331-337.
Perrin, R. M., Konopatskaya, O., Qiu, Y., Harper, S., Bates, D. O. and Churchill, A. J. (2005) Diabetic retinopathy is associated with a switch in splicing from anti- to pro-angiogenic isoforms of vascular endothelial growth factor. Diabetologia, 48, 2422-2427.
Roth T., Burger A. M., Dengler W., Willmann H. and Fiebig H. H. Human tumor cell lines demonstrating the characteristics of patient tumors as useful models for anticancer drug screening. In: Fiebig H H, Burger A M (eds). Relevance of Tumor Models for Anticancer Drug Development. Contrib. Oncol. 1999, 54: 145-156.
Ruan B, Bovee M L, Sacher M, Stathopoulos C, Poralla K, Francklyn C S, Söll D. (2005) A unique hydrophobic cluster near the active site contributes to differences in borrelidin inhibition among threonyl-tRNA synthetases. J. Biol. Chem., 280, 571-577.
Singh, S. K., Gurusiddaiah, S., and Whalen, J. W. (1985) Treponemycin, a nitrile antibiotic active against Treponema hyodysenteriae. Antimicrob. Agents Chemother. 27:239-245.
Smith M. B. and March J. (2001) March's advanced organic chemistry, 5^{th} Ed, John Wiley and Sons Inc., UK
Tsuchiya, E., Yukawa, M., Miyakawa, T., Kimura, K. I., and Takahashi, H. (2001) Borrelidin inhibits a cyclin-dependent kinase (CDK), Cdc28/Cln2, of Saccharomyces cerevisiae. J. Antibiot. 54:84-90.
Vong, B. G., Kim, S.-H., Abraham, S. and Theodorakis, E. A. (2004) Stereoselective total synthesis of (−)-borrelidin. Angew. Chem. Int. Ed., 43, 3947-3951.
Wakabayashi, T., Kageyama, R., Naruse, N., Tsukahara, N., Funahashi, Y., Kitoh, K., and Watanabe, Y. (1997) Borrelidin is an angiogenesis inhibitor; disruption of angiogenic capilla vessels in a rat aorta matrix culture model. J. Antibiot. 50:671-676.
Wakasugi, K., T. Nakano, et al. (2005). “Oxidative Stress-Responsive Intracellular Regulation Specific for the Angiostatic Form of Human Tryptophanyl-tRNA Synthetase.” Biochemistry 44(1): 225-32.

1. A compound according to Formula (I) below, or a pharmaceutically acceptable salt thereof: 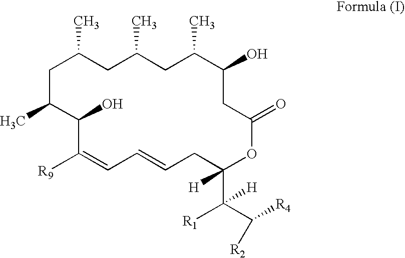
Where:
R₁ and R₂ each independently represent H, SR₃ or a C1-C4 alkyl group which may be optionally substituted with one or more groups selected from OH, F, Cl or SR₃; where R₃ represents H, CH₃ or COCH₃; alternatively R₁ and R₂ together with the carbons to which they are joined represent a 4 membered cycloalkyl ring optionally substituted with one or more halo atoms or one or more C1 to C3 alkyl groups;
R₄ represents 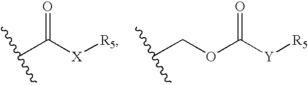
or —NHNHC(O)R₈ where R₈ represents biotin, H or a C1-C4 alkyl group optionally substituted with one or more groups selected from OH, F, Cl;
X represents —NH— or —O—;
Y represents —NH—, —O— or —CH₂—;
R₅ represents H or —(CH₂)ₙR₆,
n represents an integer between 1 and 3,
R₆ represents H, —OH, —OCH₃, —CO₂R₇, or a C1 to C4 alkyl group optionally substituted with one or more groups selected from OH, F, Cl, —CO₂R₇, —COR₇, where R₇ represents a C1-C4 alkyl group
or R₆ represents:
i) a 6 membered aromatic ring,
ii) a 5 to 7 membered heteroaromatic ring containing between one and three N, S or O atoms,
iii) a 5-7 member cycloalkyl group or
iv) a 5-7 membered heteroalkyl ring containing between one and three N, S or O atoms,
each of i) to iv) above may be optionally substituted with one or more groups selected from CH₃, OCH₃, F, Cl or Br; and
R₉ represents CN, CO₂H, CH₃ or CONH₂,
provided, however, that when X represents —O— then R₅ does not represent H.

2. The compound according to claim 1 wherein R₉ represents CN.

3. The compound according to claim 1 wherein R₁ and R₂ together with the carbons to which they are joined represent a 4 membered cycloalkyl ring.

4. The compound according to claim 3 wherein R₁ and R₂ together with the carbons to which they are joined represent a 4 membered cycloalkyl ring, only substituted by R₄.

5. The compound according to claim 1 wherein R₄ represents

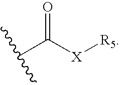

6. The compound according to claim 1 wherein X represents —O—.

7. The compound according to claim 1 wherein X represents —NH—.

8. The compound according to claim 1 wherein n represents 1.

9. The compound according to claim 1 wherein n represents 2.

10. The compound according to claim 1 wherein when R₆ represents a 6 membered heteroaromatic ring containing between one and three N, S or O atoms.

11. The compound according to claim 10 wherein R₆ represents a 6 membered heteroaromatic ring containing one N atom.

12. The according to claim 11 wherein R₆ represents

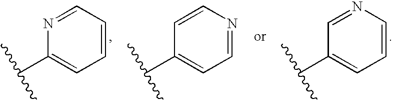

13. The compound according to claim 1 wherein R₆ represents a 6-membered heteroalkyl ring containing between one and three N, S or O atoms.

14. The compound according to claim 13 wherein R₆ represents

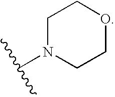

15. The compound according to claim 1 wherein R₁ and R₂ together with the carbons to which they are joined represent a 4 membered cycloalkyl ring, substituted only with R₄ which represents 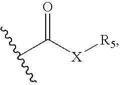
where X represents —O— and R₅ represents —(CH₂)ₙR₆, where n represents 2 and R₆ represents

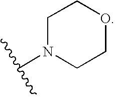

16. The compound according to claim 1 wherein R₁ and R₂ together with the carbons to which they are joined represent a 4 membered cycloalkyl ring substituted only with R₄ which represents 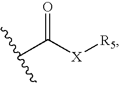
where X represents —O— and R₅ represents —(CH₂)ₙR₆, where n represents 2 and R₆ represents

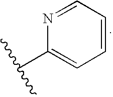

17. The compound according to claim 1 wherein R₁ and R₂ together with the carbons to which they are joined represent a 4 membered cycloalkyl ring substituted only with R₄ which represents 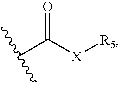
where X represents —NH— and R₅ represents —(CH₂)ₙR₆, where n represents 2 and R₆ represents

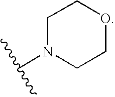

18. The compound according to claim 1 wherein R₁ and R₂ together with the carbons to which they are joined represent a 4 membered cycloalkyl ring substituted only with R₄ which represents 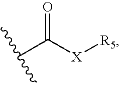
where X represents —NH— and R₅ represents —(CH₂)ₙR₆, where n represents 1 and R₆ represents

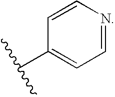

19. The compound according to claim 1 wherein R₁ and R₂ together with the carbons to which they are joined represent a 4 membered cycloalkyl ring substituted only with R₄ which represents 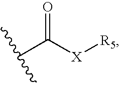
where X represents —NH— and R₅ represents —(CH₂)ₙR₆, where n represents 1 and R6 represents

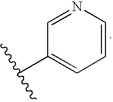

20. The compound according to claim 1 wherein R₁ and R₂ together with the carbons to which they are joined represent a 4 membered cycloalkyl ring substituted only with R₄ which represents 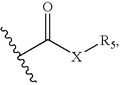
where X represents —NH— and R₅ represents —(CH₂)ₙR₆, where n represents 1 and R₆ represents

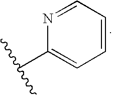

21. The compound according to claim 1 wherein R₁ and R₂ together with the carbons to which they are joined represent a 4 membered cycloalkyl ring substituted only with R₄ which represents 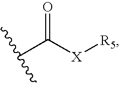
where X represents —NH— and R₅ represents —(CH₂)ₙR₆, where n represents 2 and R₆ represents

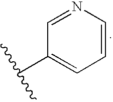

22. The compound according to claim 1 wherein R₁ and R₂ together with the carbons to which they are joined represent a 4 membered cycloalkyl ring substituted only with R₄ which represents 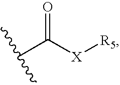
where X represents —NH— and R₅ represents H.

23. The compound according to claim 1 which is:
17-des(cyclopentane-2-carboxylic acid)-17-(cyclobutane-2-carboxylic acid)borrelidin (4-(2-hydroxyethyl))morpholine ester;
17-des(cyclopentane-2-carboxylic acid)-17-(cyclobutane-2-carboxylic acid)borrelidin (2-(2-hydroxyethyl)pyridine ester;
17-des(cyclopentane-2-carboxylic acid)-17-(cyclobutane-2-carboxylic acid)borrelidin (4-(2-aminoethyl))morpholine amide;
17-des(cyclopentane-2-carboxylic acid)-17-(cyclobutane-2-carboxylic acid)borrelidin (4-aminomethyl)pyridine amide;
17-des(cyclopentane-2-carboxylic acid)-17-(cyclobutane-2-carboxylic acid)borrelidin (3-aminomethyl)pyridine amide;
17-des(cyclopentane-2-carboxylic acid)-17-(cyclobutane-2-carboxylic acid)borrelidin (2-aminomethyl)pyridine amide;
17-des(cyclopentane-2-carboxylic acid)-17-(cyclobutane-2-carboxylic acid)borrelidin (3-(2-aminoethyl))pyridine amide;
17-des(cyclopentane-2-carboxylic acid)-17-(cyclobutane-2-carboxylic acid)borrelidin amide;
or a pharmaceutically acceptable salt of any one thereof.

24. (canceled)

25. (canceled)

26. A pharmaceutical composition comprising a compound according to claim 1 together with one or more pharmaceutically acceptable diluents or carriers.

27. A method of treatment of cancer or B-cell malignancies which comprises administering to a patient an effective amount of a compound according to claim 1.

28. (canceled)

29. A method of treatment of ophthalmic disorders in which angiogenesis is implicated which comprises administering to a patient an effective amount of a compound according to claim 1.

30. The method according to claim 29 wherein the ophthalmic disorder is diabetic retinopathy.

31. The method according to claim 29 wherein the ophthalmic disorder is age related macular degeneration, corneal neovascularisation and retinopathy of prematurity.

32. A process for preparing a compound of formula (I) or a pharmaceutically acceptable salt thereof as defined in claim 1 which comprises:
(a) reacting a compound of formula (II): 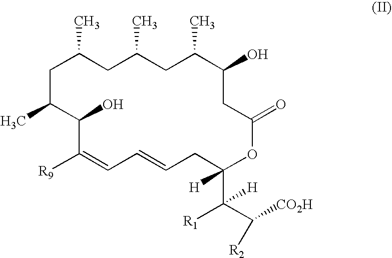
wherein R₁, R₂ and R₉ are as defined in claim 1,
or a protected derivative thereof, using any one of the methods described in (i) to (viii) below:
(i) reaction of an acid chloride formed from a compound of formula (II) with a suitable alcohol or amine;
(ii) direct esterification or amidation of a compound of formula (II) in the presence of carbodiimide, and a base;
(iii) transesterification of an ester, formed from a compound of formula (II), with a suitable alcohol;
(iv) reaction of a methyl ester of a compound of formula (II) with a suitable amine;
(v) formation of an active ester from a compound of formula (II), then reaction with a suitable alcohol or amine;
(vi) formation of a mixed anhydride from a compound of formula (II), then reaction with a suitable amine;
(vii) reduction of a mixed anhydride from a compound of formula (II) with a metal hydride to an alcohol;
(viii) alkylation and acylation of a primary alcohol prepared from a compound of formula (II); or
(b) converting a compound of formula (I) or a salt thereof to another compound of formula (I) or another pharmaceutically acceptable salt thereof; or
(c) deprotecting a protected compound of formula (I).
